(19) United States
(12) Patent Application Publication
(10) Pub. No.: US 2015/0306412 A1
(43) Pub. Date: Oct. 29, 2015

(54) Method to Provide a Variable Tesla to a Whole Body using an Electromagnetic Coil and Control

(71) Applicant: Kristi Lee Durschmidt, Chandler, AZ (US)

(72) Inventor: Kristi Lee Durschmidt, Chandler, AZ (US)

(21) Appl. No.: 14/695,569

(22) Filed: Apr. 24, 2015

Related U.S. Application Data
(60) Provisional application No. 61/983,789, filed on Apr. 24, 2014.

(51) Int. Cl. A61N 2/02

(52) U.S. Cl. CPC A61N 2/02

(57) ABSTRACT

A device for electro-magnetic care to address certain ailments of the human body by use of a varying magnetic field in proximity to the whole human body. The variable magnetic field is shaped by a current waveform which is applied to a large electromagnetic coil surrounding a human body. The device comprises an electromagnetic coil and suitable power supply to create the desired wave form and frequency suitable for an alternative medical care. The electromagnetic field is noninvasive and is applied to the entire body simultaneously.

### CROSS REFERENCE TO RELATED APPLICATIONS

This application claims the benefit of provisional application No. 61/983,789 filed on Apr. 24, 2014. The entire provisional application is incorporated herein by reference.

### STATEMENT REGARDING FEDERALLY SPONSORED RESEARCH OR DEVELOPMENT

Not applicable.

### REFERENCE TO SEQUENCE LISTING, A TABLE, OR COMPUTER PROGRAM LISTING

Not applicable.

### BACKGROUND OF THE INVENTION

(1) Field of the Invention

This invention is directed toward magnet therapies that are believed to improve a patient's health. It is designed around ‘whole body’ patient care which target particular ailments by use of magnetic fields that are generated by electrical wave forms at varying intensities and frequencies.

(2) Description of Related Art

Magnetic therapy is an alternative form of medical treatment that is believed to affect various parts and tissues of the human body, in particular, the blood vessels, and also for the reduction of inflammation of various parts of human anatomy. While it is considered an alternate treatment, many individuals will claim that this kind of therapy provides relief for various ailments such as arthritis, pains, inflammation, and provides for improved blood circulation. It is also believed that certain pathogens and body tissues will react to magnetic fields, and are advantageously affected by a close magnetic field which varies in frequency.

Others have worked in the field of magnetic therapy. For example, U.S. Pat. No. 6,280,376 describes an electromagnetic treatment. While a treatment is described, it does not cover the entire body at once. U.S. Pat. No. 5,014,699 also has similar limitations.

U.S. Pat. No. 6,234,953 describes pulsed magnetic treatments, but only at a low frequency of less than 300 Hz.

U.S. Pat. No. 7,175,587 is typical of small magnetic treatment devices, and only describes a small hand held device, which is not effective in treating the entire body all at once.

Commercial sales by ASA Laser (www.asalaser.com—PMT Qs) intend to provide for whole body treatment, but the treatment coil is too small to be effective for simultaneous, whole body treatment. When the coil is moved from position to position, the patient's blood continues to flow and some portion does not receive effective treatment. Additionally, to provide treatment at a specified length of time requires the patient to wait for the treatment at each coil position to be completed. This makes for a very long patient session.

An effective pulsed magnetic field is needed which applies the field to the whole body simultaneously, rather than particular parts, or portions at a time. An important benefit of whole body magnetic care is speed due to the ability to combine multiple sessions utilizing different frequencies, waveforms, and intensities into a single session. The time for electromagnetic care is reduced which is an important enhancement to convenience.

Pulsed electromagnetic therapy is considered to be controversial for some uses. Benefits of electromagnetic therapy for bones and bone cell repair are known and continue to be studied. Pulse electromagnetic field (PEMF) stimulators are the most commonly used type of noninvasive bone growth and spinal fusion stimulators.

Benefits of pulsed electromagnetic therapy in other tissues is controversial, and the Food and Drug Administration (FDA) has a history of issuing fines and penalizing companies that promise cures for cancer and other diseases due to a lack of scientifically approved and recognized studies, completed according to the scientific standards of the FDA. It is important that any pulsed magnetic field care fits into the FDA guidelines by not promising benefits to patients that would imply FDA approval, or obtain therapy benefits from scientific studies that are free from controversy.

A portion of the population believes there are diseases and health conditions that will respond to pulsed electromagnetic therapy. An individual may have come to the belief that their health is improved by pulsed electromagnetic therapy based on personal study or experience. Table 1 below is information that pulsed electromagnetic therapy is believed will address, according to some sources.

TABLE 1

| Hz | Benefits |
|---|---|
| 0-4 | Promotes Growth Hormone for healing and regeneration. |
| 40 | Helpful for cognition. |
| 4-12 | Helpful for learning and memory. |
| 7-12 | Helpful for deeper states of consciousness. |
| 45-67 | Mosquito Repelling Frequencies |
| 51 | Makes the sphere of energy larger |
| 73 | Helpful in acupuncture and increasing circulation in |
| | areas being treated |
| 147 | For areas scar tissue tendons, ligaments. |
| 294 | For tissue of ectodermal origin, such as body openings, |
| | skin and nerve. |
| 587 | For circulatory and lymphatic stimulation and treatment |
| 1,174 | For tissue of mesodermal origin, such as bone, joints, |
| | ligament, viscera and tendon. |
| 1,552 | Frequency for worms/parasites |
| 2,114 | Morgellons Frequency |
| 2,349 | For chronic conditions |
| 4,698 | For pain control. |
| 20,000 | General use and especially anti-aging purposes. |
| 378,000 | Borrelia - Gulf War Syndrome |
| 378,500 | Borrelia - Gulf War Syndrome alternate frequency |
| 382,000 | Borrelia - Gulf War Syndrome alternate frequency |

The ability to provide equipment that is capable of the frequencies above during any patient session, or provide any patient requested frequency, is important to respond to patient wishes. Patient sessions that apply a pulsed magnetic field will provide reassurance, contentment, and comfort to individuals that are concerned about these various ailments, and have come to believe that pulsed electro-magnetic therapy will address their health issues.

What is needed is a pulsed electromagnet system in combination with a patient session that will respond to individual concerns that the magnetic field is applied to the entire body at once.

### BRIEF SUMMARY OF THE INVENTION

The primary goal is to direct uniform inline magnetic fields into the human body that that are variable in intensity and frequency. The electromagnet disclosed in the present invention is conceived to be a large, body length ring (i.e. hollow cylinder), fully encompassing a patient, with an associated electrical control system that provides the desired magnetic field intensity at the desired frequency. The system is designed to be capable of providing multiple, simultaneous, wave forms and frequencies.

### BRIEF DESCRIPTION OF THE SEVERAL VIEWS OF THE DRAWING(S)

FIG. 1 is a side view of the patient who is inside the therapeutic electromagnetic coil.

FIG. 2 is an abbreviated version of one possible electrical control system for regulating the amount of current in the electromagnetic coil.

FIGS. 3A & 3B are plan and elevation of a patient room.

### DETAILED DESCRIPTION OF THE INVENTION

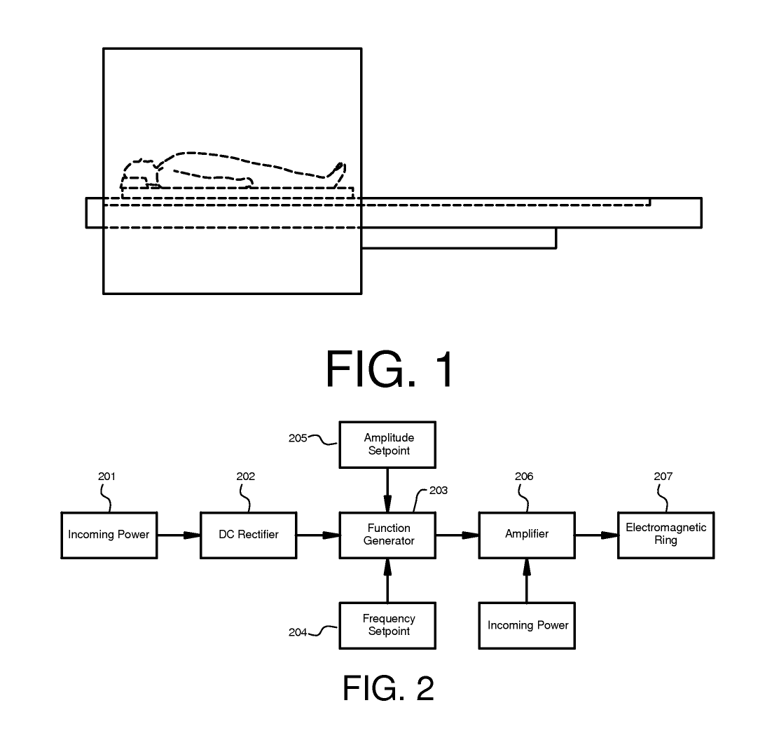

FIG. 1 is a side view of the patient who is inside the therapeutic electromagnetic coil. A preferred embodiment is to provide pulsed electromagnetic fields to the whole body as illustrated in the figure.

FIG. 2 is an electrical control system that is capable of providing the desired therapeutic magnetic field. The incoming power 201 is optionally rectified to a DC current by a rectifier 202. After that, a function generator 203 provides a sine wave output at a variable frequency and amplitude based on operator setpoints of amplitude 205 and frequency 204. This wave form is amplified 206 and the electricity then flows into the electromagnetic ring 207. To convert the incoming power to a controllable frequency and amplitude, either solid state electrical equipment or rotational equipment may be used, such as a motor-generator set. It is a preferred embodiment to use solid state equipment. The control of the function generator 203 is preferably provided for and monitored by, a computer, however this is not a strict requirement. Dial and push button settings into a hardwired control system may equally be used. Noise suppressors are optionally used on the function generator output if there is an electrical noise problem that significantly affects the magnetic field being generated.

The pulsed current waveform may be a sine wave, a square wave, a triangle wave, or a saw-tooth wave. All pulsed electromagnetic fields use these waveforms. The function generator is designed to provide any frequency for the current waveforms between 1 Hz to 11,780,000 Hz.

Alternatively, the function generator is completely self-contained as a direct ‘plug into the wall’ style, and utilizes commonly available power such as 110 VAC or 220 VAC. Such devices are known in the art, and the circuitry methods used to create the wave form are not a part of the present invention. In an alternate embodiment, an arbitrary wave form generator (AWG) is used to control the electric current into the electromagnet coil.

The function generator capability is designed based on the desired maximum magnetic field strength, which determines a maximum needed power. An optimum electromagnetic coil is also used to establish the power capability of the function generator, and winding and power losses are included in the calculation.

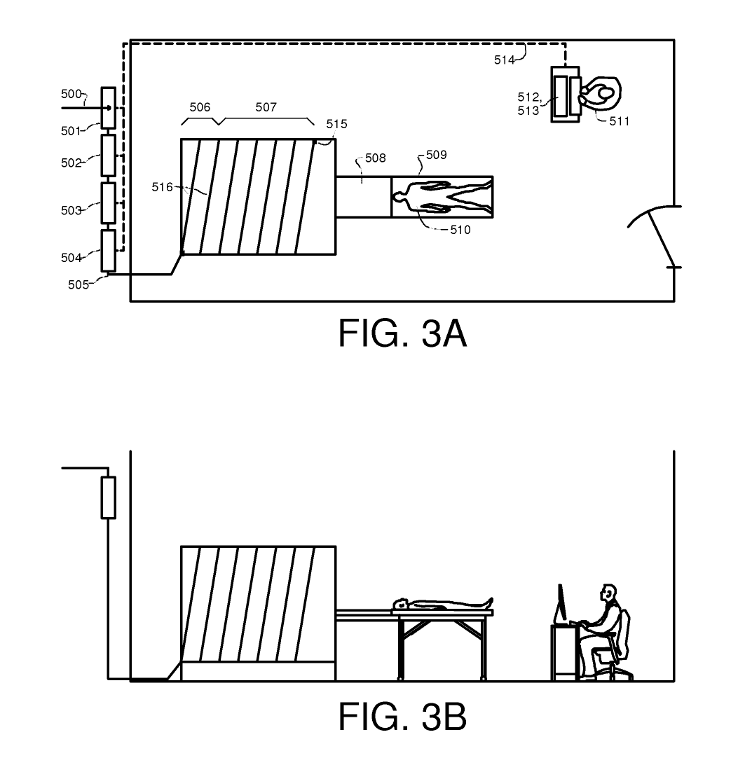

FIGS. 3A & 3B are a plan view and an elevation view of an embodied room that provides pulsed electromagnetic fields to the whole body of a patient.

The incoming power supply to the building is a typical 220 VAC and inside the building, the power supply 500 to the electrical control system is 110 vac.

The electrical supply to the electromagnet coil comes from an electrical control system, which includes a function generator. Incoming power is directed to a Tesla AC or DC rectifier/Converter/Power 501. This, in turn, feeds a frequency generator 502. An optional noise suppression is used, such as a ferrite suppressor, to remove unwanted frequencies in the output of the frequency generator. The output is then adjusted using an a second adjustable frequency converter 503 (if needed). Finally, the output from the second frequency converter is fed into an amplifier 504 which amplifies either the voltage or current.

A cable 505 from the amplifier then goes through the wall to the electromagnet coil which incorporates a scanning section 506 at the left end as viewed, and an electromagnetic care section 507 on the right as viewed. A sliding patient table 508 allows the patient to be conveniently inserted into the electro-magnetic care area 507. A patient table 509 supports the patient 510 just before going into the electro-magnetic care area. A technologist 511 operates a computer with display 512, 513 which feeds a communication cable 514 that controls the electrical supply to the electromagnet coil. The electromagnet coil utilizes insulated copper windings 516 and stops at terminal 515. The overall resistance of the cooper windings 516 (end to end) is approximately 8 ohms. Additionally, the circumferential copper wiring is designed to provide a uniform magnetic field inside the volume of the electrical magnetic coil when an electric current passes through it.

The overall length of the electromagnet coil is approximately eight feet.

If enough power is used in the electromagnet coil, an optional cooling system (not shown) is used such as a fan or water cooled tubes. It is designed to cool the circumferential wiring of the electromagnet to a designated temperature amount.

The patient 509 is moved in and out of the electromagnet coil 507 area using a table 508 which slides conveniently into (and out of) the center of the electromagnet coil. The patient is preferably centered in the electromagnet coil.

The operator station is used to control the electro-magnetic field using adjustable volts, amps, time, and output power (watts) along with adjustable timing of the application of the electro-magnetic field. A graph of the frequency used to treat the patient is also provided by a computer display/picture. After electro-magnetic care is completed, a printout is made which records the patient name, and a log of how the patient was treated (such as frequency, time, power level, etc.).

The control system is designed to be capable of adjusting the frequencies to the electromagnet coil, and is especially capable of providing a 2,114 Hz frequency, and additionally, a frequency range of 2,114 up to 382,000 Hz to get a that is believed to address nano robots, various diseases, various infections by a portion of the population. It also provides a particular frequency of 2,114 Hz, which is believed to treat Morgellons disease. It is also capable of adjusting to 382,000 Hz to address Borrelia (Gulf War Syndrome).

Important features of the disclosed invention include:
1) A electro-magnetic coil which covers the entire body, that is, from head to toe while the patient lies horizontally inside the coil.
2) A patient table that slides in and out of the electro magnet coil, and the table is capable of supporting up to a 300 lb person.
3) A sine wave is the preferred magnetic field. However, other wave forms such as a square wave, a triangle wave, or a saw-tooth wave are also used. Optionally, the wave forms are used in combination, such as the use of multiple sine waves overlapped with each wave form at a different frequency and magnitude.
4) Two stop switches (not shown in figures) are used to provide safety for the operator and patient. An operator stop (kill) switch is used to shut the magnetic field down for any reason. The patient also is given a stop switch to stop the power into the magnetic coil if the patent is uncomfortable or panics. The patient has operational control over the stop switch and it is in the patient's possession during a patient session.
5) The amplitude of the magnetic field (the field strength) is variable based on the current input into the magnetic coil. The operator control may be simplified to three different levels of electro-magnetic care: mild, medium, and strong based on the comfort of the patient and the desired level of electro-magnetic care.
6) In one embodiment, the frequency of the magnetic field is designed to provide a therapeutic frequency of 2,114 Hz, but also is variable and capable of providing higher and lower frequency electro-magnetic fields. The electro-magnetic care would optionally include an overlay of 15 Hz by overlaying a separate waveform on top of the 2,114 Hz waveform.
7) During electro-magnetic care, the patient is put into the middle of the electromagnetic coil, so that the patient is completely surrounded by the coil.
8) A convenient and comfortable moving table is used to place the patient inside the coil, to avoid the need for the patient to self move, who may not be very capable of motion.
9) A disclaimer statement that is signed by the patient which contains language that disclaims success for application of a magnetic field against any known disease or health condition,
10) The patient is required to self-determine a session criterion for the amount and type of electromagnetic field. The criterion for a patient session includes the wave form(s), the desired waveform frequency, the time length for the application of the magnetic field, and the magnetic field strength. The patient may optionally request a field strength of as strong, moderate, and mild which are taken to mean the upper ⅓, middle ⅓, and lower ⅓ of the capability of the function generator's maximum power.
11) The patient session information is re-interpreted by the operator to the type of electrical current waveform. The calculation is based on the electro-magnetic device characteristics which includes wiring resistance and magnetic field characteristics of the electromagnetic coil.
12) determining criterion for a patient session including:
   a. current frequency and amplitude for the multiple waveforms based on the circumferential wiring around the electromagnetic ring,
   b. sequence and timing of the single or multiple waveforms.
13) applying the magnetic field and completing the patient session according to items 10 -12.

While various embodiments of the present invention have been described, the invention may be modified and adapted to various operational methods to those skilled in the art. Therefore, this invention is not limited to the description and figure shown herein, and includes all such embodiments, changes, and modifications that are encompassed by the scope of the claims.

I claim:

1. The method of providing a variable electromagnetic field to the entire body of a patient according to a desire of said patient comprising:
A. providing:
a. an electrical magnetic coil in the shape of a hollow cylinder, wherein said electrical magnetic coil is long enough to cover an entire human body,
b. circumferential wiring for said electrical magnetic coil, wherein said circumferential wiring is designed to provide a uniform magnetic field in the inside volume of said electrical magnetic coil when an electric current passes through it,
c. a function generator capable of providing said electrical current, wherein said function generator is capable of providing a plurality of current waveforms,
d. wherein said function generator is capable of providing said plurality of current waveforms simultaneously into said electrical magnetic coil, wherein said current waveforms include:
i. sine wave,
ii. triangular wave,
iii. square wave, and
iv. sawtooth wave,
e. a horizontal patient table that slides in and out of said electrical magnetic coil, wherein said patient table is capable of supporting a patient weight of up to 300 lbs,
f. wherein said function generator is designed to provide any frequency for said current waveforms between 1 Hz to 11,780,000 Hz,
g. a cooling system for said circumferential wiring, wherein said cooling system for said circumferential wiring provides temperature control at a predetermined temperature value,
h. at least two stop switches that are capable of turning off said electrical current, wherein said patient has operational control over a said stop switch,
i. wherein the design of said electrical magnetic coil and said patient table provides for said patient to be placed completely within said electrical magnetic coil, and
j. a disclaimer for said patient, wherein said disclaimer contains language that disclaims success for application of a magnetic field against any known disease or health condition,
B. obtaining:
a. a patient signature on said disclaimer,
b. patient criterion for application of an electromagnetic field, and
c. wherein said patient criterion includes desired frequency, at least one wave form, time length for application for said electromagnetic field, and magnetic field strength,
C. determining criterion for a patient session including:
a. current frequency and amplitude for said multiple waveforms based on said circumferential wiring, and
b. sequence and timing of said at least one wave form,
D. positioning:
a. at least on stop switch with said patient,
b. said patient inside said electrical magnetic coil,
E. directing:
a. said function generator to be switched on, and
b. sequence and timing of said at least one wave form based on criterion for said patient session into said circumferential wiring,
and
F. completing said patient session,
whereby said electromagnetic field is applied to said patient according to said patient criterion.

2. The method according to claim 1 wherein said multiple waveform is a combination of a more than one said sine wave.

3. The method according to claim 2 wherein each said sine wave is at a different frequency and amplitude.

4. The method according to claim 1 wherein said multiple waveform is a single sine wave at a frequency of 2,114 Hz.

5. The method according to claim 1 wherein said multiple waveform is a single sine wave at a frequency of 382,000 Hz.

6. The method according to claim 1 wherein said patient criterion is divided into three levels of application of magnetic field: strong, medium, and mild, wherein said three levels of application are in the upper ⅓, middle ⅓, and lower ⅓ of the capability of said function generator's maximum power.
